(19) United States
(12) Patent Application Publication
(10) Pub. No.: US 2022/0409864 A1
(43) Pub. Date: Dec. 29, 2022

(54) INDWELLING CATHETER, MEDICAL VALVE, AND CATHETER ASSEMBLY

(71) Applicant: TERUMO KABUSHIKI KAISHA, Tokyo (JP)

(72) Inventor: Hideaki KINOSHITA, Kofu-shi (JP)

(73) Assignee: TERUMO KABUSHIKI KAISHA, Tokyo (JP)

(21) Appl. No.: 17/901,816

(22) Filed: Sep. 1, 2022

Related U.S. Application Data
(63) Continuation of application No. PCT/JP2021/008862, filed on Mar. 8, 2021.

(30) Foreign Application Priority Data
Mar. 9, 2020 (JP) 2020-039811

(51) Int. Cl. A61M 25/06; A61M 25/00

(52) U.S. Cl. CPC A61M 25/0606; A61M 25/0097; A61M 25/0693

(57) ABSTRACT

An indwelling catheter includes: a catheter configured to be indwelled in a living body; a catheter hub that holds and fixes the catheter; and a valve mechanism provided in a lumen of the catheter hub. The valve mechanism includes: a first member including a slit through which an inner needle is insertable, and a first liquid passage hole through which liquid is passable, a second member arranged adjacent to the first member and including a needle insertion hole through which the inner needle is insertable, and a second liquid passage hole through which the liquid is passable, and an operation member configured to move the first member and the second member relative to each between an open state and a closed state.

### CROSS-REFERENCE TO RELATED APPLICATIONS

This is a bypass continuation of PCT Application No. PCT/JP2021/008862, filed on Mar. 8, 2021, which claims priority to Japanese Application No. JP2020-039811, filed on Mar. 9, 2020. The entire contents of these applications are hereby incorporated by reference.

### BACKGROUND

The present disclosure relates to an indwelling catheter, a medical valve, and a catheter assembly used to perform infusion, blood transfusion and the like, for example.

### RELATED ART

Conventionally, a catheter assembly is used to perform infusion, blood transfusion and the like to a patient. This type of catheter assembly is provided with a catheter, a catheter hub fixed to a proximal end of the catheter, an inner needle inserted into the catheter, and a needle hub fixed to a proximal end of the inner needle. In the catheter assembly, after the catheter is inserted into a blood vessel of the patient, the inner needle is pulled out of the catheter and the catheter hub. Thereafter, when a medical device is connected to the catheter hub, an indwelled catheter (indwelling catheter) serves as an introduction unit.

In an operation of the indwelling catheter, there is a risk that blood flows out of the catheter hub after the inner needle is removed, and contamination by the blood or blood infection of a medical worker occurs. In order to prevent such blood outflow, a catheter assembly provided with a valve in a catheter hub is also known (JP 2014-528807 A).

### SUMMARY

Because a valve in a catheter hub is normally in a closed state, it is necessary to push a component referred to as a plug into the valve to open the valve in a case in which blood sampling, medication and the like is performed. When the plug is pushed in, a load is applied to the valve, and if this is repeatedly opened, sealability of the valve is deteriorated. Therefore, when the opening operation is repeatedly performed, there is a problem that hemostasis of the valve becomes insufficient and blood flows out of the catheter hub.

An object of one embodiment is to provide an indwelling catheter, a medical valve, and a catheter assembly capable of surely preventing a blood outflow even in a case in which a valve mechanism is opened a plurality of times.

According to one aspect of the disclosure, an indwelling catheter includes a catheter configured to be indwelled in a living body, a catheter hub that holds and fixes the catheter, and a valve mechanism provided in a lumen of the catheter hub, in which the valve mechanism includes a first member including a slit through which an inner needle is insertable and a first liquid passage hole through which liquid passes, a second member arranged adjacent to the first member and including a needle insertion hole through which the inner needle is inserted and a second liquid passage hole through which the liquid passes, and an operation member that moves the first member and the second member relative to each other, and the first member and the second member are switchable between an open state in which the first liquid passage hole and the second liquid passage hole communicate with each other and a closed state in which the first liquid passage hole and the second liquid passage hole do not communicate with each other by an operation of the operation member.

According to another aspect, a medical valve is configured to be provided in a lumen of a catheter hub that holds and fixes a catheter. The medical valve includes a first member including a slit through which an inner needle is insertable and a first liquid passage hole through which liquid passes, a second member arranged adjacent to the first member and including a needle insertion hole through which the inner needle is inserted and a second liquid passage hole through which the liquid passes, and an operation member configured to move the first member and the second member relative to each other into an open state in which the first liquid passage hole and the second liquid passage hole are communicated with each other and fluid is able to flow and a closed state in which the first liquid passage hole and the second liquid passage hole are not communicated with each other and the fluid does not flow.

According to still another aspect, a catheter assembly comprises a catheter configured to be indwelled in a living body, a catheter hub that holds and fixes the catheter, a valve mechanism provided in a lumen of the catheter hub, an inner needle inserted through the valve mechanism and inserted into the catheter, and a needle hub that holds and fixes the inner needle, in which the valve mechanism includes a first member including a slit through which the inner needle is insertable and a first liquid passage hole through which liquid passes, a second member arranged adjacent to the first member and including a needle insertion hole through which the inner needle is inserted and a second liquid passage hole through which the liquid passes, and an operation member configured to move the first member and the second member relative to each other into an open state in which the first liquid passage hole and the second liquid passage hole are communicated with each other and fluid is able to flow and a closed state in which the first liquid passage hole and the second liquid passage hole are not communicated with each other and the fluid does not flow.

According to the indwelling catheter, medical valve, and catheter assembly in the above-described aspect, it is possible to surely prevent the blood outflow even in a case in which the valve mechanism is opened a plurality of times.

### BRIEF DESCRIPTION OF DRAWINGS

FIG. 1 is a perspective view of a catheter assembly according to a first embodiment;

FIG. 2 is a perspective view of a catheter hub of the catheter assembly in FIG. 1;

FIG. 3 is a cross-sectional view of the catheter hub in FIG. 2;

FIG. 4A is a perspective view of a distal end side of a first member in FIG. 3, and FIG. 4B is a perspective view of a proximal end side of the first member;

FIG. 5A is a perspective view of a distal end side of a second member in FIG. 3, and FIG. 5B is a perspective view of a proximal end side of the second member;

FIG. 6A is an explanatory view illustrating rotation positions of the first member and the second member in a closed state of a valve mechanism, and FIG. 6B is an explanatory view illustrating rotation positions of the first member and the second member in an open state of the valve mechanism;

FIG. 7 is a perspective view of a catheter hub at the time of administration; and

FIG. 8 is a cross-sectional view of the catheter hub in FIG. 7.

### DETAILED DESCRIPTION

Preferred embodiments of an indwelling catheter, a medical valve, and a catheter assembly are hereinafter described in detail with reference to the accompanying drawings. Note that dimensional ratios in the drawings may be exaggerated and different from actual ratios for convenience of description.

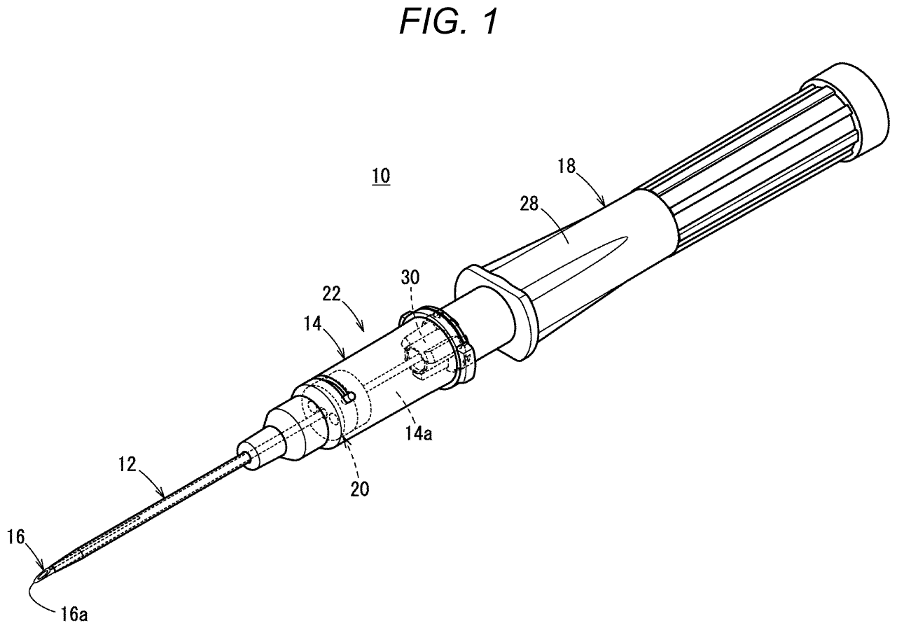

A catheter assembly 10 is provided with a catheter 12, a catheter hub 14 connected to a proximal end of the catheter 12, an inner needle 16 including a sharp cutting edge 16a at a distal end thereof, the inner needle 16 inserted into the catheter 12, a needle hub 18 connected to the inner needle 16, and a valve mechanism 20 (medical valve) arranged in the catheter hub 14 as illustrated in FIG. 1.

The catheter assembly 10 is configured such that the needle hub 18 is gripped by a user (doctor, nurse and the like), and a blood vessel of a patient is punctured with a distal end part thereof. In an initial state before use (before puncture of the patient), the catheter assembly 10 has a double tube structure in which the inner needle 16 is inserted into the catheter 12, and the inner needle 16 protrudes from a distal end of the catheter 12 by a predetermined length.

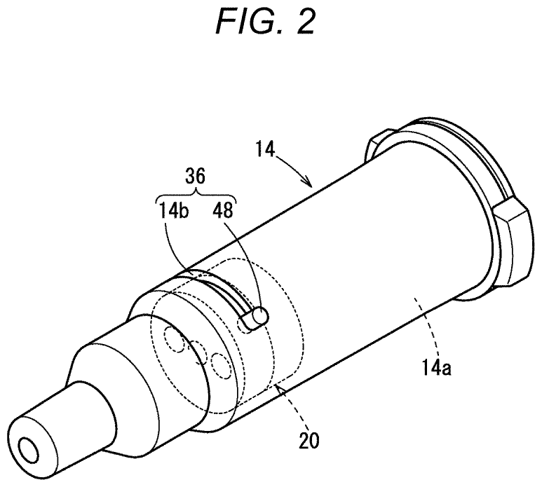

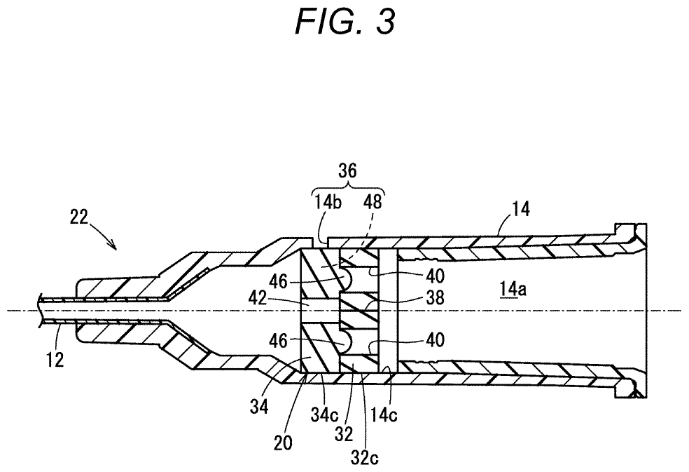

The catheter 12 is a small-diameter tubular member having flexibility. The catheter 12 is formed of, for example, a resin material, preferably a soft resin material. A hollow catheter hub 14 is connected to be fixed to the proximal end of the catheter 12 as illustrated in FIG. 1. As illustrated in FIGS. 2 and 3, the valve mechanism 20 is provided in a lumen 14a of the catheter hub 14. The catheter 12, the catheter hub 14, and the valve mechanism 20 form an indwelling catheter 22 as illustrated in FIG. 1.

When the catheter assembly 10 is used, the catheter hub 14 is exposed on a skin in a state in which the catheter 12 is indwelled in the blood vessel, and is adhered to the skin with a dressing material, a tape and the like to be placed thereon.

The inner needle 16 is a tubular member having rigidity capable of puncturing the skin of the patient, and is formed of, for example, a metal material (stainless steel) and the like. On an upper surface of a distal end part of the inner needle 16, a groove for checking blood flashback may be formed. In the initial state of the catheter assembly 10 illustrated in FIG. 1, a proximal end part of the inner needle 16 is arranged in the lumen 14a of the catheter hub 14. The inner needle 16 may also be formed as a solid needle.

The needle hub 18 is connected to the proximal end part of the inner needle 16. The needle hub 18 includes a hub main body 28 that serves as a grip gripped by the user, and a needle holder 30 that protrudes from the hub main body 28 in a distal direction and holds the proximal end part of the inner needle 16. In the initial state of the catheter assembly 10, the needle holder 30 is inserted into a proximal end part of the catheter hub 14.

The valve mechanism 20 illustrated in FIG. 3 has a function of preventing a blood outflow from the catheter hub 14 due to a blood backflow in a proximal direction, and a function of allowing passage of a liquid medicine in the distal direction at the time of liquid medicine administration. The valve mechanism 20 is provided with a first member 32 made of an elastic material, a second member 34 arranged adjacent to the first member 32, and an operation member 36 that rotates the second member 34 with respect to the first member 32.

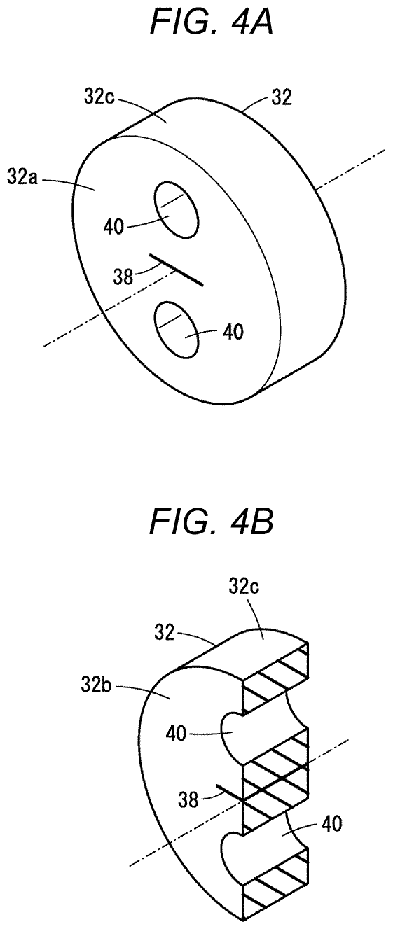

As illustrated in FIG. 4A, the first member 32 is formed into a cylindrical shape, and an outer diameter thereof is formed to be substantially as large as an inner diameter of an inner peripheral surface 14c (refer to FIG. 3) of the catheter hub 14. A distal end face 32a perpendicular to a central axis of the first member 32 is formed on a distal end side of the first member 32. As illustrated in FIG. 4B, a proximal end face 32b perpendicular to the central axis of the first member 32 is formed on a proximal end side of the first member 32. The first member 32 is formed of rubber materials such as, for example, synthetic rubber such as polybutadiene series, nitrile series, and chloroprene series, natural rubber such as polyisoprene, or thermosetting elastomer such as urethane rubber, silicon rubber, and fluorine rubber, thermoplastic elastomer, or other elastomers. As illustrated in FIG. 3, the first member 32 is pushed into the inner peripheral surface 14c of the catheter hub 14 while being elastically compressed. An outer side surface 32c of the first member 32 is in close contact with the inner peripheral surface 14c, and the first member 32 closes the inner peripheral surface 14c in a liquid-tight and air-tight manner.

As illustrated in FIG. 4A, a slit 38 is formed in the vicinity of the central axis of the first member 32. As illustrated in FIG. 4B, the slit 38 is formed to penetrate the first member 32 in an axial direction, and the inner needle 16 (refer to FIG. 1) and the like may be inserted through the same in the axial direction. The slit 38 closed by an elastic restoring force of the first member 32 may allow the insertion of the inner needle 16 therethrough while preventing the blood backflow. The slit 38 is formed into a linear shape as seen from the front, but the shape is not limited thereto; the slit may have various shapes such as a shape in which both ends are branched into a Y shape or an X shape.

A first liquid passage hole 40 is formed in a portion separated from the central axis of the first member 32. The first liquid passage hole 40 is formed to penetrate the first member 32 in the axial direction, and may allow liquid such as blood or a medicine to pass therethrough in the axial direction. As illustrated in FIG. 4A, a pair of first liquid passage holes 40 is provided with the central axis interposed therebetween. The number of the first liquid passage holes 40 may be one or three or more.

As illustrated in FIG. 3, the second member 34 is arranged adjacent to the distal end side of the first member 32. A central axis (rotation axis) of the second member 34 is arranged on the same axis as the central axis of the first member 32 and the central axis of the catheter hub 14. The second member 34 is attached so as to be rotatable about the axis of the catheter hub 14, and is rotatable relative to the first member 32.

The second member 34 is made of a material harder than that of the first member 32. More preferably, when this is formed of a material that easily slides with respect to the catheter hub 14, a rotation operation becomes smooth, and operability is preferably improved. Examples of the material of the second member 34 include various resin materials such as polypropylene resin, for example.

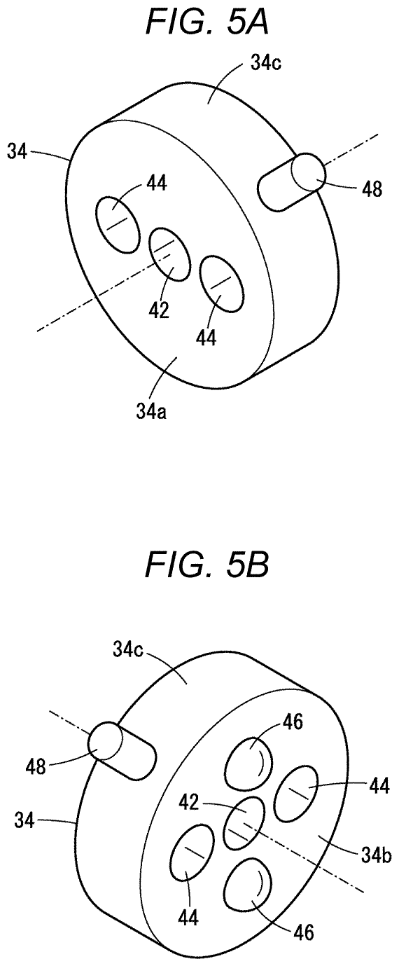

As illustrated in FIG. 5A, the second member 34 is formed into a cylindrical shape, and an outer diameter thereof is formed to be substantially as large as the inner diameter of the inner peripheral surface 14c of the catheter hub 14. A distal end face 34a perpendicular to the central axis is formed on a distal end side. As illustrated in FIG. 5B, a proximal end face 34b perpendicular to the central axis is formed on a proximal end side. A needle insertion hole 42 is formed in the vicinity of the central axis of the second member 34. The needle insertion hole 42 is formed to penetrate the second member 34 in the axial direction, and the inner needle 16 may be inserted through the same in the axial direction.

A second liquid passage hole 44 is formed in a portion separated from the central axis of the second member 34. The second liquid passage hole 44 is formed in the same position in a radial direction as the first liquid passage hole 40 of the first member 32, and communicates with the first liquid passage hole 40 when the second member 34 is rotated to a predetermined position. The second liquid passage hole 44 is formed to penetrate the second member 34 in the axial direction, and may allow the liquid such as the blood or medicine to pass therethrough by communicating with the first liquid passage hole 40. A pair of second liquid passage holes 44 is provided with the central axis interposed therebetween as is the case with the first liquid passage holes 40. The number of the second liquid passage holes 44 may be one or three or more. In order to secure a flow rate (liquid passage flow rate) of the liquid that passes therethrough, an inner diameter of the second liquid passage hole 44 is formed to be equal to or larger than an inner diameter of the first liquid passage hole 40.

The proximal end face 34b of the second member 34 is a surface facing the distal end face 32a of the first member 32, and is formed as a flat surface slidable with respect to the distal end face 32a. The proximal end face 34b is provided with a pair of protrusions 46 in positions in which they are insertable into the first liquid passage holes 40. The second liquid passage holes 44, in a quantity at least as great as that of the first liquid passage holes 40, are also provided in positions in which they are insertable in to the first liquid passage holes 40. The protrusion 46 is formed into a hemispherical shape having a diameter slightly larger than the inner diameter of the first liquid passage hole 40, and when the protrusion 46 is inserted into the first liquid passage hole 40, the protrusion 46 fits into the first liquid passage hole 40 to seal the first liquid passage hole 40 in a liquid-tight manner. In this manner, in this embodiment, the first liquid passage hole 40 is sealed by the protrusion 46, so that the proximal end face 34b of the second member 34 is not required to be in close contact with the distal end face 32a of the first member 32 in a liquid-tight manner.

An operation lever 48 is provided on an outer side surface 34c of the second member 34. The operation lever 48 is formed into a bar shape extending radially outward from the second member 34. The operation lever 48 is inserted into a guide groove 14b of the catheter hub 14 (refer to FIG. 2), and a part of the operation lever 48 protrudes out of the catheter hub 14 via the guide groove 14b. The guide groove 14b is formed to extend over a predetermined angle range in a circumferential direction of the catheter hub 14, and is configured to guide a rotation operation of the second member 34 via the operation lever 48. The guide groove 14b and the operation lever 48 form the operation member 36 of this embodiment.

As illustrated in FIG. 3, the outer side surface 34c of the second member 34 is in close contact with the inner peripheral surface 14c of the catheter hub 14 in a liquid-tight manner, so that the liquid such as the blood does not leak from the proximal end side or the guide groove 14b through an outer side of the outer side surface 34c.

The catheter assembly 10, the valve mechanism 20 (medical valve), and the indwelling catheter 22 of this embodiment are formed as described above, and an action thereof is hereinafter described.

When the catheter assembly 10 is used, the user (doctor, nurse and the like) grips the needle hub 18 of the catheter assembly 10 in the initial state illustrated in FIG. 1 and punctures the blood vessel of the patient with the distal end parts of the inner needle 16 and the catheter 12. After the puncture, the user advances the catheter 12 by pushing the catheter hub 14 in the distal direction while maintaining positions of the needle hub 18 and the inner needle 16, thereby inserting the catheter 12 into the blood vessel.

After the catheter 12 is inserted into the blood vessel by a predetermined length, the user next pulls the needle hub 18 in the proximal direction in a state in which a position of the indwelling catheter 22 is maintained, and removes the inner needle 16 from the indwelling catheter 22. At that time, the inner needle 16 is removed from the valve mechanism 20. As a result, the indwelling catheter 22 is indwelled on a patient side. After pulling the inner needle 16 out of the indwelling catheter 22, the user fixes the catheter hub 14 to the patient with the dressing material, tape and the like.

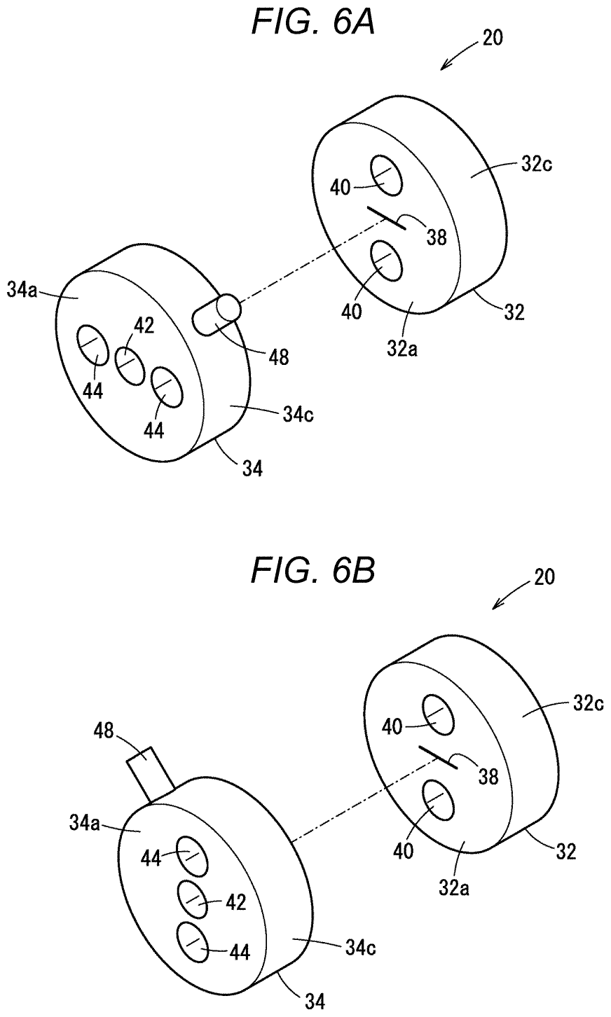

The valve mechanism 20 is provided in the catheter hub 14, and the blood outflow from the catheter hub 14 is prevented even after the inner needle 16 is removed from the valve mechanism 20. In the initial state, the first member 32 and the second member 34 of the valve mechanism 20 are arranged as illustrated in FIG. 6A, and are in a closed state in which the first liquid passage hole 40 of the first member 32 and the second liquid passage hole 44 of the second member 34 do not communicate with each other. At that time, as illustrated in FIG. 3, the slit 38 of the first member 32 is closed in a liquid-tight manner by the elastic restoring force. The protrusion 46 of the second member 34 is inserted into the first liquid passage hole 40, and the first liquid passage hole 40 is closed in a liquid-tight manner by the second member 34. In this manner, the blood outflow is prevented by the valve mechanism 20.

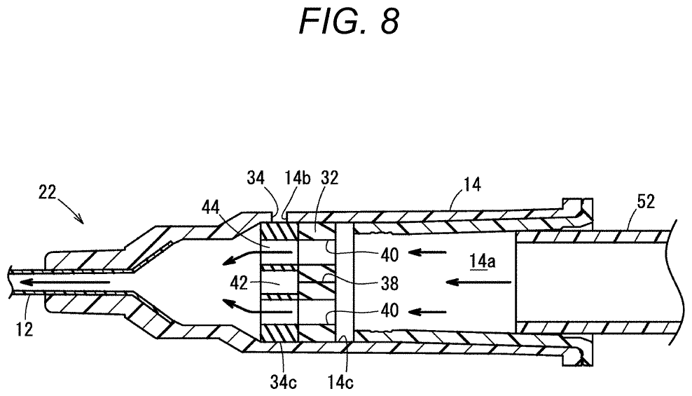

Next, as illustrated in FIG. 8, a connector 52 of an infusion tube is inserted into the lumen 14a of the catheter hub 14 on a proximal end side of the indwelling catheter 22 from which the inner needle 16 is removed, and the liquid medicine is administered from the infusion tube to the patient.

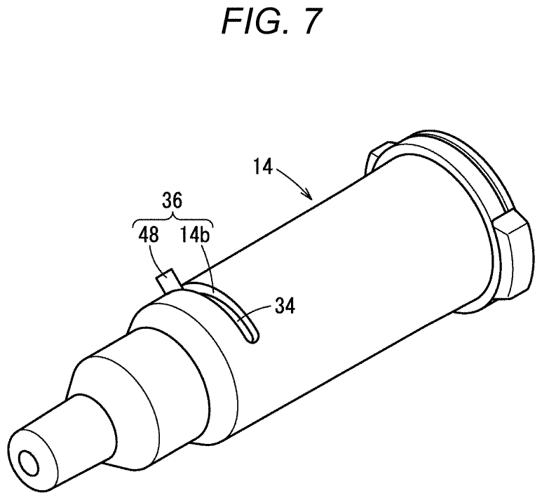

After connecting the infusion tube to the catheter hub 14, the user rotates the operation lever 48 to a position illustrated in FIG. 7 along the guide groove 14b to put the valve mechanism 20 into an open state. In the open state of the valve mechanism 20, as illustrated in FIG. 6B, positions of the first liquid passage hole 40 of the first member 32 and the second liquid passage hole 44 of the second member 34 in a circumferential direction coincide with each other. As a result, as illustrated in FIG. 8, the first liquid passage hole 40 and the second liquid passage hole 44 communicate with each other, and the liquid may flow. At that time, the protrusion 46 elastically deforms the first member 32 in a portion other than the first liquid passage hole 40. A gap through which the liquid may pass is formed between the first member 32 and the second member 34 by the protrusion 46, and a part of the liquid may flow through the gap and the needle insertion hole 42. As a result, the valve mechanism 20 is opened, and the flow of the liquid medicine from a proximal end side to a distal end side of the valve mechanism 20 is allowed.

After the administration of the liquid medicine is completed, the user closes the valve mechanism 20 by rotating the operation lever 48 to a position in FIG. 6A. The user may switch the opening and closing of the valve mechanism 20 by rotating the operation lever 48 as necessary at the time of blood sampling, medication and the like. When switching the opening and closing of the valve mechanism 20, it is not necessary to push a plug into a slit structure of an elastic member as in a conventional check valve, so that it is possible to prevent deterioration in sealability of the valve mechanism 20. Therefore, according to the valve mechanism 20 of the embodiment, the blood outflow may be prevented even when an opening/closing operation is performed a plurality of times.

The catheter assembly 10, the valve mechanism 20 (medical valve), and the indwelling catheter 22 of this embodiment exhibit the following effect.

The indwelling catheter 22 of this embodiment is provided with the catheter 12 indwelled in a living body, the catheter hub 14 that holds and fixes the catheter 12, and the valve mechanism 20 (medical valve) provided in the lumen 14a of the catheter hub 14. The valve mechanism 20 of the indwelling catheter 22 includes the first member 32 including the slit 38 through which the inner needle 16 may be inserted and the first liquid passage hole 40 through which the liquid passes, the second member 34 arranged adjacent to the first member 32 and including the needle insertion hole 42 through which the inner needle 16 is inserted and the second liquid passage hole 44 through which the liquid passes, and the operation member 36 that moves the first member 32 and the second member 34 relative to each other, and switches by the operation of the operation member 36 between the open state in which the first liquid passage hole 40 and the second liquid passage hole 44 are moved relative to each other so that the first liquid passage hole 40 and the second liquid passage hole 44 are communicated with each other and the fluid may flow, and the closed state in which the first liquid passage hole 40 and the second liquid passage hole 44 are not communicated with each other and the fluid does not flow.

According to the above-described configuration, the opening and closing may be performed only when the valve mechanism 20 moves the first member 32 and the second member 34 relative to each other. As a result, because it becomes not necessary to push the plug into the elastic member for sealing when the valve is put into the open state as in the conventional check valve, the sealability of the valve mechanism 20 may be maintained even when the opening/closing operation is performed a plurality of times. This makes it possible to surely prevent the blood outflow even when the user performs the opening/closing operation of the valve mechanism 20 a plurality of times for blood sampling, medication and the like. Because the plug and an operation mechanism thereof as in the conventional check valve become unnecessary, the structure is simplified, and a manufacturing cost may be reduced.

In the indwelling catheter 22 described above, the first member 32 may be made of the elastic material and fixed to the catheter hub 14, and the second member 34 may be displaced between the open state and the closed state by rotating with respect to the first member 32. In this manner, by making the second member 34 having relatively higher slidability than the first member 32 rotatable, the opening/closing operation may be smoothly performed, and operability is improved.

In the indwelling catheter 22 described above, the needle insertion hole 42 and the slit 38 may be provided in the vicinity of the rotation axis of the second member 34, and the first liquid passage hole 40 and the second liquid passage hole 44 may be provided in positions separated from the rotation axis of the second member 34. With this configuration, the inner needle 16 may be inserted through the needle insertion hole 42 and the slit 38 regardless of a rotational position of the second member 34. The communication and closing of the first liquid passage hole 40 and the second liquid passage hole 44 may be switched by the rotation of the second member 34, and the structure of the valve mechanism 20 is simplified.

In the indwelling catheter 22 described above, the operation lever 48 as a part of the operation member 36 may protrude out of the catheter hub 14, and the second member 34 may be configured to be rotatable from the outside of the catheter hub 14. With this configuration, the user may easily perform the opening/closing operation of the valve mechanism 20.

In the indwelling catheter 22 described above, the outer side surfaces 32c and 34c of the first member 32 and the second member 34, respectively, may be in contact with the inner peripheral surface 14c of the catheter hub 14 in a liquid-tight manner. With this configuration, it is possible to prevent the blood outflow through the inner peripheral surface 14c of the catheter hub 14.

In the indwelling catheter 22 described above, the second member 34 may include the protrusion 46 on the proximal end face 34b facing the first member 32, and the protrusion 46 may fit into the first liquid passage hole 40 of the first member 32 in the closed state to be able to close the first liquid passage hole 40 in a liquid-tight manner. With this configuration, the sealability of the first liquid passage hole 40 is improved, and the blood outflow may be more effectively prevented.

In the indwelling catheter 22 described above, the first member 32 and the second member 34 may be in close contact with each other in the axial direction. Since the first member 32 and the second member 34 are in close contact with each other, the sealability in a state in which the first liquid passage hole 40 and the second liquid passage hole 44 do not coincide with each other is preferably improved.

In the indwelling catheter 22 described above, the first liquid passage hole 40 and the second liquid passage hole 44 may be provided in positions separated from the rotation axis of the second member 34 by the same distance in the radial direction, the positions that coincide with each other when the second member 34 is rotated. As a result, by rotating the second member 34, the first liquid passage hole 40 and the second liquid passage hole 44 surely communicate with each other, and a sufficient liquid passage flow rate may be secured.

In the indwelling catheter 22 described above, the inner diameter of the second liquid passage hole 44 may be formed to be equal to or larger than the inner diameter of the first liquid passage hole 40. Such a configuration is preferable because the sufficient liquid passage flow rate may be secured.

The valve mechanism 20 (medical valve) of this embodiment is provided in the lumen 14a of the catheter hub 14 that holds and fixes the catheter 12. The valve mechanism 20 (medical valve) includes the first member 32 including the slit 38 through which the inner needle 16 may be inserted and the first liquid passage hole 40 through which the liquid passes, the second member 34 arranged adjacent to the first member 32 and including the needle insertion hole 42 through which the inner needle 16 is inserted and the second liquid passage hole 44 through which the liquid passes, and the operation member 36 that moves the first member 32 and the second member 34 relative to each other, and switches by the operation of the operation member 36 between the open state in which the first liquid passage hole 40 and the second liquid passage hole 44 are communicated with each other and the fluid may flow, and the closed state in which the first liquid passage hole 40 and the second liquid passage hole 44 are not communicated with each other and the fluid does not flow.

According to the valve mechanism 20 in the above-described aspect, it is possible to surely prevent the blood outflow even in a case in which the valve mechanism 20 is opened a plurality of times.

The catheter assembly 10 of this embodiment relates to the catheter assembly 10 provided with the catheter 12 indwelled in the living body, the catheter hub 14 that holds and fixes the catheter 12, the valve mechanism 20 provided in the lumen 14a of the catheter hub 14, the inner needle 16 inserted through the valve mechanism 20 and inserted into the catheter 12, and the needle hub 18 that holds and fixes the inner needle 16. The valve mechanism 20 of the catheter assembly 10 includes the first member 32 including the slit 38 through which the inner needle 16 may be inserted and the first liquid passage hole 40 through which the liquid passes, the second member 34 arranged adjacent to the first member 32 and including the needle insertion hole 42 through which the inner needle 16 is inserted and the second liquid passage hole 44 through which the liquid passes, and the operation member 36 that moves the first member 32 and the second member 34 relative to each other, and switches by the operation of the operation member 36 between the open state in which the first liquid passage hole 40 and the second liquid passage hole 44 are communicated with each other and the fluid may flow, and the closed state in which the first liquid passage hole 40 and the second liquid passage hole 44 are not communicated with each other and the fluid does not flow.

According to the catheter assembly 10 in the above-described aspect, it is possible to surely prevent the blood outflow even in a case in which the valve mechanism 20 is opened a plurality of times.

(Other Variations)

This embodiment is not limited to the above-described example, and the following modifications may be made.

Although the first member 32 is arranged on the proximal end side and the second member 34 is arranged on the distal end side in the example illustrated in FIG. 3, it is also possible to arrange the first member 32 on the distal end side and the second member 34 on the proximal end side.

Instead of forming the operation member 36 of the operation lever 48 extending from the outer side surface 34c of the second member 34 and the guide groove 14b of the catheter hub 14, a part of the catheter hub 14 may be configured to be rotatable in the circumferential direction, and the second member 34 may be configured to rotate integrally with a rotating portion of the catheter hub 14.

It is not required to provide the protrusion 46 on the proximal end face 34b of the second member 34. In this case, the sealability in the closed state between the first liquid passage hole 40 and the second liquid passage hole 44 may be maintained by energizing the second member 34 and the first member 32 in the axial direction to be brought into close contact with each other in a liquid-tight manner.

The indwelling catheter, the medical valve (valve mechanism), and the catheter assembly are described above with reference to preferred embodiments, but the present invention is not limited to the above-described embodiments, and it goes without saying that various modifications may be made without departing from the gist of the present invention.

What is claimed is:

1. An indwelling catheter comprising:
a catheter configured to be indwelled in a living body;
a catheter hub that holds and fixes the catheter; and
a valve mechanism provided in a lumen of the catheter hub: wherein:
the valve mechanism comprises:
a first member including a slit through which an inner needle is insertable, and a first liquid passage hole through which liquid is passable,
a second member arranged adjacent to the first member and including a needle insertion hole through which the inner needle is insertable, and a second liquid passage hole through which the liquid is passable, and
an operation member configured to move the first member and the second member relative to each between an open state in which the first liquid passage hole and the second liquid passage hole communicate with each other and a closed state in which the first liquid passage hole and the second liquid passage hole do not communicate with each other.

2. The indwelling catheter according to claim 1, wherein:
the first member is made of an elastic material and fixed to the catheter hub, and the second member is configured to be displaced between the open state and the closed state by rotating with respect to the first member.

3. The indwelling catheter according to claim 2, wherein:
the needle insertion hole and the slit are provided in the vicinity of a rotation axis of the second member, and the first liquid passage hole and the second liquid passage hole are provided in positions separated from the rotation axis of the second member.

4. The indwelling catheter according to claim 2, wherein:
a part of the operation member protrudes out of the catheter hub, and the second member is configured to be rotated from the outside of the catheter hub.

5. The indwelling catheter according to claim 4, wherein:
outer side surfaces of the first member and the second member are in contact with an inner peripheral surface of the catheter hub in a liquid-tight manner.

6. The indwelling catheter according to claim 1, wherein:
the second member comprises a protrusion on a surface facing the first member, and the protrusion fits into the first liquid passage hole of the first member to close the first liquid passage hole in a liquid-tight manner in the closed state.

7. The indwelling catheter according to claim 1, wherein:
the first member and the second member are in close contact with each other in an axial direction.

8. The indwelling catheter according to claim 2, wherein:
the first liquid passage hole and the second liquid passage hole are located in positions separated from the rotation axis of the second member by the same distance in a radial direction, the positions coinciding with each other when the second member is rotated.

9. The indwelling catheter according to claim 1, wherein:
an inner diameter of the second liquid passage hole is equal to or larger than an inner diameter of the first liquid passage hole.

10. A medical valve configured to be provided in a lumen of a catheter hub that holds and fixes a catheter, the medical valve comprising:
a first member including a slit through which an inner needle is insertable, and a first liquid passage hole through which liquid is passable;
a second member arranged adjacent to the first member and including a needle insertion hole through which the inner needle is insertable, and a second liquid passage hole through which the liquid is passable; and
an operation member configured to move the first member and the second member relative to each between an open state in which the first liquid passage hole and the second liquid passage hole communicate with each other and a closed state in which the first liquid passage hole and the second liquid passage hole do not communicate with each other.

11. A catheter assembly comprising:
a catheter configured to be indwelled in a living body;
a catheter hub that holds and fixes the catheter;
a valve mechanism provided in a lumen of the catheter hub;
an inner needle inserted through the valve mechanism and inserted into the catheter; and
a needle hub that holds and fixes the inner needle, wherein:
the valve mechanism comprises:
a first member including a slit through which the inner needle is insertable, and a first liquid passage hole through which liquid is passable,
a second member arranged adjacent to the first member and including a needle insertion hole through which the inner needle is insertable, and a second liquid passage hole through which the liquid is passable, and
an operation member configured to move the first member and the second member relative to each between an open state in which the first liquid passage hole and the second liquid passage hole communicate with each other and a closed state in which the first liquid passage hole and the second liquid passage hole do not communicate with each other.
